(19) United States
(12) Patent Application Publication
(10) Pub. No.: US 2016/0158308 A1
(43) Pub. Date: Jun. 9, 2016

(54) TREATMENT OF MULTIPLE EVOLVING BACTERIAL RESISTANCE DISEASES WITH LIPOSOMALLY FORMULATED GLUTATHIONE

(71) Applicants: CHILDREN'S HEALTHCARE OF ATLANTA, INC., Atlanta, GA (US); YOUR ENERGY SYSTEMS, LLC, Palo Alto, CA (US); EMORY UNIVERSITY, Atlanta, GA (US)

(72) Inventors: Lou Ann Brown, Atlanta, GA (US); Frederick Timothy Guilford, Palo Alto, CA (US)

(73) Assignees: EMORY UNIVERSITY, Atlanta, GA (US); CHILDREN'S HEALTHCARE OF ATLANTA, INC., Atlanta, GA (US); YOUR ENERGY SYSTEMS, LLC, Palo Alto, CA (US)

(21) Appl. No.: 14/768,148

(22) PCT Filed: Aug. 14, 2014
(86) PCT No.: PCT/US2014/051083

§ 371 Date: Aug. 14, 2015

Related U.S. Application Data
(60) Provisional application No. 61/903,038, filed on Nov. 12, 2013.

(51) Int. Cl. A61K 38/06; A61K 31/59; A61K 9/127

(52) U.S. Cl. CPC A61K 38/063; A61K 9/127; A61K 31/59

(57) ABSTRACT

The invention proposes routine treatment of patients, particularly those admitted to an ICU, with Vitamin D and liposomal glutathione for prophylaxis and treatment against Group B Streptococcus and Carbapenem-resistant enterobacteriaceae, and biomarkers to measure effectiveness of treatment. Further, because the method of treatment bolsters body defenses as well as appearing to have direct killing action, the propensity to create more and more antibiotic- and/or carbapenem resistant strains is downgraded.

### TECHNICAL FIELD

### Statement of Industrial Applicability

The invention relates to the use of liposomally formulated reduced glutathione as a simultaneous prophylaxis and to treat carbapenem-resistant enterobacteriaceae resistant diseases having evolving bacterial resistance and to treat diseases such as Group B streptococcus, especially in neonates, having evolving bacterial resistance.

### BACKGROUND

Carbapenem-resistant Enterobacteriaceae (CRE) are a very difficult problem which the inventor believes is accelerating in difficulty because of the potential of many CRE to evolve protective mechanisms against antibiotics. The Centers for Disease Control and Prevention in the United States (“CDC”) has stated:
“CRE, which stands for carbapenem-resistant Enterobacteriaceae, are a family of germs that are difficult to treat because they have high levels of resistance to antibiotics. Klebsiella species and Escherichia coli (E. coli) are examples of Enterobacteriaceae, a normal part of the human gut bacteria, that can become carbapenem-resistant. Types of CRE are sometimes known as KPC (Klebsiella pneumoniae carbapenemase) and NDM (New Delhi Metallo-beta-lactamase). KPC and NDM are enzymes that break down carbapenems and make them ineffective. Both of these enzymes, as well as the enzyme VIM (Verona Integron-Mediated Metallo-β-lactamase) have also been reported in Pseudomonas.
Healthy people usually do not get CRE infections—they usually happen to patients in hospitals, nursing homes, and other healthcare settings. Patients whose care requires devices like ventilators (breathing machines), urinary (bladder) catheters, or intravenous (vein) catheters, and patients who are taking long courses of certain antibiotics are most at risk for CRE infections.
Some CRE bacteria have become resistant to most available antibiotics. Infections with these germs are very difficult to treat, and can be deadly—one report cites they can contribute to death in up to 50% of patients who become infected.” www(dot) cdc (dot) gov/hai/organisms/cre accessed Aug. 12, 2014.

For instance, Klebsiella pneumonia is a growing issue in intensive care units as it is a common bacterial contaminant that has become relatively refractory to current treatment regimes. Additionally, because Klebsiella tends to be omnipresent in the body, though well controlled, the intensive care unit of a hospital or medical care facility functions as an incubator for strains of Klebsiella that have shown an increasing pattern of producing ever more antibiotic resistant strains of Klebsiella pneumoniae in patients. Further those strains appear to have a gene characteristic that enables the transfer of the increased antibiotic resistance to other virulent and more widespread diseases which this invention is also designed to curtail. The CDC amplifies its earlier discussion stating:
“Common Enterobacteriaceae include Klebsiella species and Escherichia coli (E. coli). These germs are found in normal human intestines (gut). Sometimes these bacteria can spread outside the gut and cause serious infections, such as urinary tract infections, bloodstream infections, wound infections, and pneumonia. Enterobacteriaceae can cause infections in people in both healthcare and community settings . . . .
Some CRE bacteria have become resistant to most available antibiotics. Infections with these germs are very difficult to treat, and can be deadly—one report cites they can contribute to death in up to 50% of patients who become infected.
Many people with CRE will have the germ in or on their body without it producing an infection. These people are said to be colonized with CRE, and they do not need antibiotics for the CRE. If the CRE are causing an infection, the antibiotics that will work against it are limited but some options are often available. In addition, some infections might be able to be treated with other therapies, like draining the infection. Bacterial strains that have been resistant to all antibiotics are very rare but have been reported.”

Treatment options are limited. The CDC describes them as follows:
“What are some things hospitals are doing to prevent CRE infections?
To prevent the spread of CRE, healthcare personnel and facilities can follow infection-control precautions provided by CDC. These include:
   Washing hands with soap and water or an alcohol-based hand sanitizer before and after caring for a patient
   Carefully cleaning and disinfecting rooms and medical equipment
   Wearing gloves and a gown before entering the room of a CRE patient
   Keeping patients with CRE infections in a single room or sharing a room with someone else who has a CRE infection
   Whenever possible, dedicating equipment and staff to CRE patients
   Removing gloves and gown and washing hands before leaving the room of a CRE patient
   Only prescribing antibiotics when necessary
   Removing temporary medical devices as soon as possible
   Sometimes, hospitals will test patients for these bacteria to identify them early to help prevent them from being passed on to other patients
What can patients do to prevent CRE infections?
Patients should:
   Tell your doctor if you have been hospitalized in another facility or country.
   Take antibiotics only as prescribed.
   Expect all doctors, nurses, and other healthcare providers wash their hands with soap and water or an alcohol-based hand rub before and after touching your body or tubes going into your body. If they do not, ask them to do so.
   Clean your own hands often, especially: Before preparing or eating food
   Before and after changing wound dressings or bandages
   After using the bathroom
   After blowing your nose, coughing, or sneezing
   Ask questions. Understand what is being done to you, the risks and benefits.
What if I have CRE?
Follow your healthcare provider's instructions. If your provider prescribes you antibiotics, take them exactly as instructed and finish the full course, even if you feel better. Wash your hands, especially after you have contact with the infected area and after using the bathroom. Follow any other hygiene advice your provider gives you.”
For providers, the references are equally grim as to lack of treatment:
“‘2012 CRE Toolkit—Guidance for Control of Carbapenem-resistant Enterobacteriaceae (CRE)’
Enterobacteriaceae, a frequent cause of both community- and healthcare-associated infections, can become resistant to carbapenems through various mechanisms, including the production of carbapenemases. Klebsiella pneumoniae carbapenemase (KPC) is the most common carbapenemase in the United States and has spread throughout many regions.
Although the prevalence of CRE likely varies from region to region, a review of data from the National Healthcare Safety Network found that in 2009-2010, about 13% of Klebsiella species reported from central line-associated bloodstream infections (CLABSIs) and catheter-associated urinary tract infections (CAUTIs) were carbapenem-nonsusceptible. About 2% of Escherichia coli reported from CLABSIs and CAUTIs were carbapenem-nonsusceptible.
Genes coding for KPC can be transmitted between bacteria via mobile genetic elements, potentially facilitating transmission of these organisms. Metallo-beta-lactamase-producing organisms (NDM, VIM, and IMP) have also been identified in the United States but appear to be less common than KPC-producing organisms” Carbapenem-Resistant Enterobacteriaceae Infection: New Guidelines, Dantes R, Guh, A, CDEC Expert Commentary, MedScape News, Jun. 21, 2012.

More broadly, the invention applies to carbapenem-resistant enterobacteriaceae strains of bacteria selected from the family groups of Klebsiella, Staphylococcus, Clostridium, Shigella, Leishmaniasis, Pneumonia, Escheria, Chlamydia, and Anthrax, on the same principle because strains of these diseases also have the ability to evolve their resistance to antibiotic treatment. The invention can act as a prophylaxis and treatment for multiple diseases simultaneously.

Group B Streptococcus (GBS) is a bacterial organism that is found normally in the gastrointestinal tract and genital tract. It can be found in 20-40% of women and is considered a commensal organism. A commensal organism is one which may benefit from the host, but has no damaging effect to the host. At the same time, GBS is a major medical concern as infection with this organism can be harmful to mother and child in childbirth related infection. GBS was originally termed Streptococcus agalactiae (lack of lactation) as it is associated with mastitis (of udders) in cows. In humans it can be a cause of postpartum infection and as the most common cause of neonatal sepsis.

Neonatal infection with GBS has been found to have a higher incidence in preterm neonates has a high mortality (4/30 preterm infants), as compared to term infants (0.24/1000 live births; mortality, 1/68 infants) (Berardi A, Rossi C, Lugli L, Creti R, Bacchi Reggiani M L, et al. Group B streptococcus late-onset disease: 2003-2010. Pediatrics. February 2013; 131(2):e361-8).

Neonatal sepsis may present either early or late after birth. Early onset occurs 85% of the time within 24 hours. Onset is most rapid in premature neonates. Late onset infection is defined as occurring at 4-90 days of life. Both early and late on set infections are more common in premature infants.

The organisms most associated with early onset sepsis in infants after birth are:

Group B Streptococcus (GBS)

Escherichia coli

Coagulase-negative Staphylococcus

Haemophilus influenzae

Listeria monocytogenes

The organisms most associated with early onset sepsis in infants after birth are:

Coagulase-negative Staphylococcus

Staphylococcus aureus

E coli

Klebsiella

Pseudomonas

Enterobacteriaceae

Candida

GBS

Serratia

Acinetobacter

Anaerobes

Pneumonia is more common in early-onset sepsis, whereas meningitis and bacteremia are more common in late-onset sepsis. Vigorous treatment for suspected neonatal sepsis is advised because neonate's relative immunosuppression.

While many of the organisms on the list of potential causes of sepsis can be effectively treated with antibiotics, there is a growing awareness that many organisms such as GBS are developing resistance to antibiotics. So, methods that provide antibiotic activity are increasingly in demand.

Additionally, because GBS is often present in the body, though well controlled, the intensive care unit of a hospital or medical care facility functions as an incubator for strains of GBS that have shown an increasing pattern of producing ever more antibiotic resistant strains of GBS in patients. Further those strains appear to have a gene characteristic that enables the transfer of the increased antibiotic resistance to other virulent and more widespread diseases which this invention is also designed to curtail.

More broadly, the invention applies to infections in, but not limited to

Group B Streptococcus (“GBS” or “Strep B”)

Escherichia coli

Coagulase-negative Staphylococcus

Haemophilus influenzae

Listeria monocytogenes

Staphylococcus aureus

E coli

Klebsiella

Pseudomonas

Enterobacteriaceae

Candida

GBS

Serratia

Acinetobacter

Anaerobes

### SUMMARY OF INVENTION

### Technical Problem

The technical problem is finding and enabling a composition for treatment to diminish the growth of CRE's and Group B Streptococcus B (“GBS” or “Strep B”) in the patient's body, particularly in lung tissue, and both bacterial and viral pneumonia more generally. This includes addressing multiple CRE diseases and Strep B simultaneously. Further, the technical problem is to use a composition that interrupts the cycle of incubation of ever more resistant strains of CRE and/or Strep Bin patients surviving infection. It is also important for any treatment to have markers to determine the efficacy promptly and the inventor proposes a composition for treatment of CRE in conjunction with using liposomally formulated reduced glutathione to treat pneumonia and in particular pneumonia Klebsiella and other CRE diseases and Strep B.

### Solution to Problem

For patients being admitted to the Intensive Care Unit at a hospital, the inventor proposes routine administration of liposomally formulated reduced glutathione (“liposomal glutathione” or “liposomal reduced glutathione”) according to the specifications below in combination with Vitamin D, particularly Vitamin D-3, Vitamin D2 or Vitamin D-25 (OH) (collectively “Vitamin D”), for treatment of CRE diseases, including Klebsiella pneumonia, and pneumonia more generally, and Strep B. Less effective but also possible is using plain glutathione in combination with vitamin D. The enablements herein would improve response to any form of infection, but are particularly directed to CRE diseases, including klebsiella, and to Strep B. Proposed biomarkers of efficacy in conjunction with the use of liposomal glutathione and Vitamin D for CRE diseases and/or Strep B include reduced glutathione levels in the plasma, bronchoalveolar lavage fluid or lung tissue and reduction from elevated levels of TGF-β. The determinative biomarker is finding the CRE or Strep B in the blood indicating sepsis, and/or finding the DNA of the CRE or Strep B in the blood or other body fluids and tissues. TGF-β sampling in blood may be particularly useful in monitoring for late onset infection in infants. PCR (polymerase chain reaction) studies to identify DNA sequences for determination of the presence of bacteria in blood or tissue samples such as lung secretion (sputum or lavage of the bronchioles and alveolae during bronchoscopy) can also be used as markers for identifying bacterial infection.

### Advantageous Effects of Invention

The invention is intended as a prophylactic and as a treatment or composition for treatment. Efficacy of treatment can be determined using the biomarkers. The composition of the invention, liposomal glutathione, has been recently shown to have utility for having an antibiotic like effect on Klebsiella pneumonia cultures in vitro, and in vivo as demonstrated by efficacy in reducing by large multiples the presence of colonies of Klebsiella in rats in animal tests. Further, because the liposomal glutathione bolsters body defenses as well as appearing to have direct killing action, the propensity arising from surviving Strep B or CRE bacteria, including Klebsiella bacteria, to create more and more resistant strains to antibiotic treatment is downgraded. Efficacy of treatment can be determined using the biomarkers.

### DESCRIPTION OF EMBODIMENTS

The purpose of the present application is to reference the use of liposomally encapsulated reduced glutathione as method of treating CRE diseases, including Klebsiella and as a means of preventing and reversing the formation of cultures of Klebsiella. More broadly, the invention applies to anti-biotic resistant strains of bacteria selected from the family groups of Klebsiella, Staphylococcus, Streptococcus B, Clostridium, Shigella, Leishmaniasis, Pneumonia, Escheria, Chlamydia, and Anthrax, on the same principle because strains of these diseases also have the ability to evolve their resistance to antibiotic treatment. As referenced in the following quotation, what drives the need for this invention is that many of these diseases are present in the body in non-threatening form; it is the decline in health coupled with the activity of the ICU that triggers the more troublesome aspects of CRE diseases.

“Klebsiella pneumonia is traditionally described as a clinically significant opportunistic bacterial pathogen that can infect immunocompromised individuals who are hospitalized and suffer from underlying diseases. In addition to it being a significant clinical pathogen, K. pneumoniae is also a normal component of the upper respiratory and GI-tract microbiota of both humans and mice. [f.n. omitted] Lau, H et al, “Host and microbiota factors that control Klebsiella pneumoniae mucosal colonization in mice” Microbes Infect. 2008 October (10) 12-13: 1283-1290 (2008) PMC 2633640.

The purpose of the present application is also to reference the use of liposomally encapsulated reduced glutathione as method of treating GBS and as a means of preventing and reversing the formation of cultures of GBS. Vitamin D as set forth herein may also be administered. More broadly, the invention applies to, but is not limited to

Group B Streptococcus (GBS)

Escherichia coli

Coagulase-negative Staphylococcus

Haemophilus influenzae

Listeria monocytogenes

Staphylococcus aureus

E coli

Klebsiella

Pseudomonas

Enterobacteriaceae

Candida

GBS

Serratia

Acinetobacter

Anaerobes

GBS is a normal vaginal commensal in up to 30% of the population antenatal treatment with oral penicillin does not reduce the likelihood of GBS colonisation at the time of delivery, and so is not required

a note of the presence of vaginal carriage should be made in the woman's maternity notes, so health professionals can take appropriate action during labor

Research has shown that the use of N-Acetyl Cysteine (NAC), a building block of glutathione can have the effect of reversing the oxidative stress in cells. A lack of adequate glutathione in the defensive immune cells such as macrophages can lead to serious deficits in immune defense against infection as related in the discussion of Venketaraman and Brown studies below. However as explained below, NAC requires the function of the enzymes needed to combine the three amino acids of glutathione as well as energy to formulate intracellular glutathione, which energy is often not available in a cell. The inventor believes a compromise of energy and enzyme function occurs because of oxidative stress induced by infections such as Klebsiella. Cysteine, as found in NAC has been the only possible oral method, however inefficient, to increase glutathione though it is not particularly effective and no showing has been made of in vivo application. Non-formulated glutathione itself, as a tripeptide, does not survive passage of the gut very well to be as physiologically effective to individual cells such as in lung tissue. Liposomally encapsulated reduced glutathione, the present invention, has been shown by Lauver et al to raise glutathione levels in tissues after oral ingestion in a rabbit model of ischemia (low oxygen) followed by the return of blood flow and oxygen (i.e., reperfusion) injury.

Vitamin D facilitates the activity of liposomally encapsulated reduced glutathione in certain aspects of the cycle in which body glutathione is generated and to facilitate the appropriate enzyme action in conjunction with glutathione and its creation.

Research was commissioned at the University of Michigan, showing the surprising effect of the invention in reversing and controlling oxidative stress in tissues such as that which occurs in individuals with illnesses severe enough to require Intensive Care Unit (ICU) admission might have.

The results of that study show that contrary to the usual degradation in the gut, the invention, purchased from Your Energy Systems, LLC of Palo Alto, Calif., in the amount of approximately 428.8 mg of GSH administered in 5 ml doses, had the following abstracted result:
“A liposomal preparation of glutathione (lipGSH) capable of oral administration was investigated for its ability to attenuate tissue injury and increase myocardial glutathione levels in an isolated heart model of reperfusion injury. Male, New Zealand white rabbits were assigned randomly among four groups: control and daily oral administration of lipGSH for three, seven or fourteen days. At completion of the dosing regimen, hearts were harvested and perfused in a retrograde manner with the use of a Langendorff apparatus. The hearts were subjected to 30 min of global ischemia followed by 60 min of reperfusion. Hearts from lipGSH-treated rabbits exhibited better recovery of left ventricular contractile function during reperfusion and had attenuated oxidative damage. Furthermore, hearts from lipGSH-treated animals had increased myocardial tissue levels of GSH demonstrating effective absorption of lipGSH.”

Lauver D A, Kaissarian N M, Lucchesi B R, Oral pretreatment with liposomal glutathione attenuates reperfusion injury in rabbit isolated hearts, J Cardiovasc Pharmacol. 2013 March; 61(3):233-9. PMID 23188132 (Lauver).

Based on the Lauver et al research, the administration of liposomally encapsulated glutathione pursuant to the invention would raise the level of intracellular glutathione by at least 30%, particularly in tissues oxidatively stressed.

Plain, non-formulated glutathione used orally is not as good an option for this therapy as plain glutathione is not absorbed well after oral ingestion in humans. A rat study of the removal of a radio-tagged metal (CO-60) from the liver, performed at Pacific Northwest National Laboratory with oral liposomally encapsulated reduced glutathione confirms this observation. The tissue from the control animals (water) served as the 100% of the toxin remaining in the tissue. The animals receiving:
a. Control (water only) showed 100% of the toxin remained=0% removal
b. Plain glutathione, oral, in water showed 100% of the toxin remained=0% removal.
c. Intravenous glutathione showed 36% of the toxin remaining=64% removal.
d. Liposomal reduced glutathione showed 53% of the toxin remaining=47% removal.

The data from this study is consistent with the observation that liposomally encapsulated glutathione is almost as effective as intravenous plain glutathione in removing the toxin. The plain glutathione has less absorption or efficacy. Levitskaia et al, Aminothiol Receptors for Decorporation of Intravenously Administered ⁶⁰Co In The Rat, Health Physics, Vol. 98(1) No. 4: 53-60 (Health Physics Society 2009).

More recently, it has been observed that resistance to antibiotic materials has been increasing. For instance, Carbapenem-Resistant Klebsiella pneumoniae (CRKP). is resistant to almost all available antimicrobial agents, Over the past 10 years, a progressive increase in CRKP has been seen worldwide. The Centers for Disease Control have asserted that the proportion of enterobacteriaceae resistant to a powerful class of antibiotics known as carbapenems—often the last resort for severe infections—increased to 4.2% in 2011 from 1.2% a decade earlier. The relative increase for the most worrisome type of organism, Klebsiella, was even greater, with 10.4% resistance to treatment compared with 1.6% a decade earlier. Dubbed CRE—for carbapenem-resistant enterobacteriaceae—the germs kill up to half of patients who get bloodstream infections from them.

Carbapenems are a class of β-lactam antibiotics with a broad spectrum of antibacterial activity. Carbapenems have a structure that renders them highly resistant to most β-lactamases.

Carbapenems are one of the antibiotics of last resort for many bacterial infections, such as Escherichia coli (E. coli) and Klebsiella pneumoniae. Recently, alarm has been raised over the spread of drug resistance to carbapenem antibiotics among these coliforms, due to production of the New Delhi metallo-β-lactamase, NDM-1. There are currently no new antibiotics in the pipeline to combat bacteria resistant to carbapenems, and worldwide spread of the resistance gene is considered a potential nightmare scenario. The acuity of the problem and non-obviousness of any current solution is highlighted in a recent BBC News article, Roberts, M, Q&A, Antibiotic Resistance, available only on-line at www(dot)bbc(dot)co(com)uk/news/health-21739378. It begins with the alarming observation: “Antibiotic resistance is developing at such a pace that we may soon face a future without cures for infection, England's Chief Medical Officer Professor Dame Sally Davies is warning. Her apocalyptic report likens the threat to that of terrorism.”

This invention proposes a method to arrest the growth of antibiotic resistant organisms by instead enhancing the body's immune systems so as to be more fully cooperative and functional with antibiotics, and to ally the development of the self-transformation aspects of bacteria which allows otherwise commensal organisms to become dangerous infections and their selection to more antibiotic resistant varieties. http: [forward double slashes]www(dot)bbc(dot)co(dt)uk[forward slash]news[forward slash]health-21739378.

Oral liposomally encapsulated reduced glutathione that is uniquely designed to be absorbed a) across the mucosa of the nose, mouth, gastrointestinal tract, b) after topical application for transdermal, or c) by intravenous infusion of glutathione with or without liposome encapsulation is prepared under the method and according to the composition described as follows:

### Basic Dosing Information

For a typical adult ranging from 55 kg to 90 kg, the dose of oral liposomally encapsulated reduced glutathione is oral liposomally encapsulated reduced glutathione 422 mg (1 teaspoon) (5 ml each) of concentration of approximately 8.25% w/w or 84 mg/ml at least twice a day. More preferable is administration of 4 teaspoons (5 ml each) 4 times per day. If tolerated well, a loading dose of another teaspoon (5 ml) after perhaps an hour would be helpful.

In a patient who is intubated for ventilator support, monitoring of the individual's pulmonary function and resistance to mechanical ventilation can be monitored as an indication of need for additional liposomal reduced glutathione. Increased airway resistance occurs during constriction of the small airways due to loss of bronchodilation and worsens the ability to adequately support the lung function of the patient. As the individual's lung tissue level of glutathione improves due to therapy with liposomal reduced glutathione improvement in bronchial diameter known as bronchodilation will allow increased lung function. A similar improvement will be identified due improvement in the local lung tissue inflammation due to increased immune cell function. The improvement in bronchodilation will be observed with both liposomal reduced glutathione and liposomally encapsulated GSNO.

Intravenous treatment can be over an extended period in the dosages referenced herein given per day in continuous or multiple parts.

There also can be additional enhancement of the liposomally encapsulated function of killing of GBS, or CRE, including Klebsiella during infection by combining administration of the referenced invention with the administration of arginine either orally or intravenously in doses of 500 to 1000 mg 1 to 4 times a day.

There also can be additional combination with the administration of Vitamin D3 or synthetic analogue of Vitamin D3 or vitamin D2 intravenously, intramuscularly, or orally in doses from 5000 IU to 100,000 IU or higher. This combination allows increased function of the enzyme glutathione reductase to regenerate glutathione that has been “used” as an antioxidant and is then in the form of oxidized glutathione (abbreviated “GSSG”) back into reduced glutathione abbreviated GSH.

The Vitamin D (250H) range of levels is 30-100 ng/ml.

Reduced Glutathione (GSH) level in plasma range is reduced glutathione: 3.8-5.5 mmol/L The combination of reduced liposomal glutathione combined with vitamin D is proposed, either synthetic or natural, or sunlight therapy to raise vitamin D to a patient critically ill in an ICU setting and monitored with biomarker testing for Vitamin D (25OH), Glutathione levels in serum should be monitored and additional doses of both liposomal glutathione and/or plain intravenous glutathione should be administered every 4 hours until the patient's condition or plasma level has returned to normal. Additional monitoring includes measurements of serum Transforming growth factor β (TGF β) as well as monitoring the patient's lung function in terms of airway resistance as an indicator of the constriction of the airways and the ability of the airways to allow the normal flow of oxygen and CO2 flow across the alveolar membranes. This can be done by comparing the level of inspired oxygen with the blood level of oxygen, conventionally known as the “blood gas levels”.

Additional benefit for the management of the severely ill patients is provided by the infusion of plain, non-encapsulated reduced glutathione. While research shows that liposomal glutathione has significant advantage (100×) over plain glutathione in terms of absorption into cells with phagocytic function such as macrophages and astrocytes (Zeevalk 2010), the direct application of plain glutathione will be helpful in suppressing the Klebsiella and other organisms that may be free floating, that is not inside of the cells of the host. Zeevalk, G et al, “Liposomal glutathione provides maintenance of intracellular glutathione and neuroprotection in mesencephalic neuronal cells,” Neurochem Research, 2010: October 35 (10), 1575-1587 Epub Jun. 10, 2010 (PMID 21463600).

If plain oral glutathione must be used because of cost, the suggested dose is 1000-5000 mg for a typical 70 kg person. Preferable would be this dose 3 times per day. Patient response would be improved somewhat with the simultaneous administration of Vitamin D as set forth in this application.

The study by Morris at Western University investigated the effect of N-acetyl cysteine (NAC) and liposomally encapsulated glutathione to prevent the replication of intracellular Mycobacterium tuberculosis after infecting the cells with the organism. Previous work by Venketaraman has shown that raising glutathione levels with NAC in this cell culture model will limit the growth of Mycobacterium tuberculosis (TB). The study shows that both NAC and liposomally encapsulated glutathione were able to limit the growth of the organisms to a level below 1000 colony forming units per milliliter (CFU/ml). NAC at 10 millimolar reduced the CFU/ml to 8,000, while the liposomally encapsulated glutathione at 5 micromolar concentration reduced the CFU/ml to 6,000 CFU/ml. This data demonstrates that liposomally encapsulated glutathione is over 2000 times more potent than NAC in maintaining the function of macrophages undergoing the oxidative stress of an intracellular infection.

The study shows that liposomally encapsulated reduced glutathione formulated per this invention has a significantly increased absorption and function in the macrophages from individuals with HIV that are undergoing infection with M. tb (Mycobacterium tuberculosis). The absorption of the liposomally encapsulated glutathione is 1000×'s more efficient than the glutathione precursor N-acetyl cysteine (NAC) in restoring normal glutathione levels and restoring the glutathione related function of slowing the replication of M tb in macrophages taken from individuals with HIV . . . “Glutathione Supplementation Improves Immune Function in HIV+ Macrophages,” Morris D, Guerra C, Khurasany M, Guilford T, Venketaraman V, J Interferon Cytokine Res. 2013 May; 33(5):270-9. PMID Epub 2013 February 14 (Western University of Health Sciences, Pomona, Calif. 91766, USA) PMID 23409922 (“Morris D”).

The surprising and novel finding in the Morris D et al study of the dramatic absorption of liposomally encapsulated reduced glutathione compared to N-acetyl cysteine (“NAC”) explains the ability of this formulated form of liposomally encapsulated reduced glutathione to restore macrophage function back to the M1 function.
“In a previous study we observed elevated levels of TGF-β in both the plasma and macrophage culture supernatants of HIV+ macrophages [42]. This elevated TGF-β will compromise the amount of GCLC present inside the cell; consequently, supplementing the raw materials [such as with NAC] for de novo synthesis in HIV+ individuals who are over expressing TGF-β will not result in the same increased production of reduced GSH that is observed in individuals who are not over expressing TGF-β. In addition, this phenomenon may explain why lGSH [the liposomally encapsulated reduced glutathione of this invention] at lower concentrations than NAC is more effective at raising the concentration of reduced GSH in HIV+ macrophages than in HIV− macrophages. Supplementing with an lGSH formulation provides complete GSH molecules to cells, circumventing the enzymatic pathway responsible for GSH production, without the requirement for the cell to construct the tripeptide. This may also explain why treatment with lGSH seems to raise the ratio of reduced GSH to GSSG at much lower concentrations than NAC, as cells treated with NAC will have to produce new molecules of reduced GSH utilizing their own enzymatic machinery. [emphasis added, citation omitted].” Morris et al.

The ability to maintain cell function by raising glutathione directly during an infectious process in the cell is novel and has not been previously reported. The observation that liposomally encapsulated glutathione is 2000 (two-thousand) times more effective in maintaining glutathione and the ability of the cell to limit replication of an intracellular infectious agent such as TB is also novel and previously unreported.

Upon entry to an intensive care unit or other site where acute care is being provided to a severely ill patient, prophylactic doses of firstly a) intravenous

GSH, in plain or in liposomal formulation in doses of 1,000 mg to 10,000 mg, b) liposomal GSH in doses of 1,000 to 10,000 milligrams or c) orally administrable glutathione according to the dosages below and secondly, vitamin D in doses from 10,000 to 100,000 IU should be administered. Subsequent doses of liposomal glutathione and/or plain glutathione should be administered every 4 hours as discussed above. A topical formulation is also possible to administer glutathione.

Severe illnesses may be precipitated by viral infections such as HIV, influenza or Coronavirus, and present as Severe acute respiratory syndrome (SARS). This situation will require assisted ventilation and during this time a state of immune compromise may develop which leaves the individual susceptible to bacterial infection such as the hospital acquired infection including Klebsiella, a CRE disease or GBS.

For management of CRE diseases referenced hereunder, including Klebsiella, related severe infection or sepsis, or GBS, it is preferably proposed that there be intravenous infusion of 1 gm. of liposomal reduced glutathione every 4 hours for a total of 6 grams per day. The 6 grams intravenous dosing may be modified into different infusion schedules as determined by the circumstances of the patient. That is, the total of 6 grams total amount may be divided into 1.5 gms. every 6 hours if needed.

In these latter situations, the ability to maintain glutathione as a direct, active agent preventing CRE or Klebsiella replication, or GBS replication will be useful. Additionally, the administration of Vitamin D3 or its synthetic derivatives such as Vitamin D2 may be useful in maintaining the function of the enzyme glucose-6 phosphodiesterase known as G6PD. This enzyme is needed for support of the enzyme glutathione reductase, which regenerates oxidized glutathione (GSSG) to reduced glutathione (GSH).

An additional combination is the simultaneous administration of the referenced liposomal reduced glutathione in combination with antibiotics known to have an effect on non-resistant forms of Klebsiella, CRE diseases, or GBS in order to improve the efficacy of the antibiotic. The compromise of bacterial function will cause a decreased function in the bacterial needed to metabolize and remove the antibiotic, causing the CRE organism, GBS or Klebsiella organism to become susceptible to the antibiotic. The ability of the administration of liposomal reduced glutathione in combination with antibiotic will increase the susceptibility of a range of organisms known to be resistant to antibiotics and will include GBS and CRE diseases but not be limited to (1) Klebsiella pneumoniae, (2) Klebsiella ozaenae, (3) Klebsiella terrigena, (4) Klebsiella rhinoscleromatis, (5) Klebsiella oxytoca, (6) Klebsiella planticola, and (7) Klebsiella ornithinolytica.

Bacteria such as GBS and CRE diseases and Klebsiella and related bacteria possess the ability to transfer DNA via bacterial conjugation, transduction or transformation, which allows genetic material to spread horizontally through an existing population. This process led to the spread of the gene encoding shiga toxin from Shigella to E. coli O157:H7, carried by a bacteriophage thereby increasing the virulence and active infectious nature of the agent. Similarly, these organisms such as GBS and CRE diseases or Shigella or E. coli, or Klebsiella can transfer the genes responsible for providing the metabolic resistance to antibiotics found in antibiotic resistant Klebsiella or the CRE organism or GBS. The administration of liposomally reduced glutathione in combination with currently available antibiotics to resistant strains of bacteria will decrease the resistance of the organism and increase the effectiveness of antibiotic therapy. The implication is that the patient can pass the resistant genes from the relatively rare resistant Klebsiella to more widespread organisms such as Shigella, or E. coli or an often sexually transmitted disease: Chlamydia, and to other GBS and CRE disease organisms.

By way of example, but not limited to the following examples, the liposomal reduced glutathione can be used alone or in combination with antibiotics for the treatment of antibiotic resistant organisms of the Group B Streptococcus genus, Klebsiella genus, Staphylococcus genus, and also for the Escherichia genus including coli, and to diminish the effects of Shigella and shigellosis, Chlamydia, and leishmaniasis. The application of the present invention diminishes replication of the Klebsiella organism whether or not it has classical antibiotic resistance or not. In the absence of liposomal glutathione, the partially or completely antibiotic resistant organism can self-replicate freely.

Klebsiella is a commensal organism in the gastrointestinal tract, but is normally constrained by mechanisms that are not well understood. GBS is often found in the vaginal area and the rectal area. By restoring natural glutathione levels using the invention where normal glutathione function is otherwise compromised, the organisms are restrained from replication, and in particular, replication to resistant forms. Commensal organisms that appear to be included in the direct action of this invention include Klebsiella, Staphylococcus genus, Streptococcus genus, and Escherichia genus including coli. Other organisms introduced into the body subject to the same phenomenon include Shigella and shigellosis, Chlamydia, and leishmaniasis.

Staphylococcus genus species include but are not limited to aureus

S. aureus group—S. aureus, S. simiae
S. auricularis group—S. auricularis
S. carnosus group—S. carnosus, S. condimenti, S. massiliensis, S. piscifermentans, S. simulans
S. epidermidis group—S. capitis, S. caprae, S. epidermidis, S. saccharolyticus
S. haemolyticus group—S. devriesei, S. haemolyticus, S. hominis
S. hyicus-intermedius group—S. chromogenes, S. felis, S. delphini, S. hyicus, S. intermedius, S. lutrae, S. microti, S. muscae, S. pseudintermedius, S. rostri, S. schleiferi
S. lugdunensis group—S. lugdunensis
S. saprophyticus group—S. arlettae, S. cohnii, S. equorum, S. gallinarum, S. kloosii, S. leei, S. nepalensis, S. saprophyticus, S. succinus, S. xylosus
S. sciuri group—S. fleurettii, S. lentus, S. sciuri, S. stepanovicii, S. vitulinus
S. simulans group—S. simulans
S. warneri group—S. pasteuri, S. warneri

The invention has further application for the treatment of methicillin resistant staphylococcus aureus, commonly referred to as MRSA. MRSA is term used to refer to staphylococcus aureus bacteria resistant to beta lactam antibiotics typically thought of as the penicillins and cephalosporins.

Other indications for which treatment is appropriate by liposomal reduced glutathione or for which administering liposomal reduced glutathione is beneficial include signs of infection that may be bacterial such as high fevers, increased white blood count on blood sample testing, findings consistent with pneumonia on physical examination, severe illness requiring administration to the hospital of intensive care unit for conditions that may be accompanied by infection, the finding of low reduced glutathione on blood testing or similar indication of oxidant stress.

The concentration of the glutathione in the liposomes can be in a range from 3.3% w/w to 9% w/w or higher. The amount of 3.3% w/w is equivalent to a concentration of 123 mM. Deionized water can be used to bring w/w percentages up to 100% w/w in any of the tables or formulations below.

Antibiotics Cooperating with Liposomal Reduced Glutathione

In conjunction with the dosing herein, the following antibiotics may be utilized by a practitioner of the medical arts of ordinary skill. The dosages of the antibiotics would be the currently prescribed dose on the package insert approved by the U.S. Food and Drug Administration (referred to as the “recommended course of administration”).

These antibiotics would include, but not be limited to:

### 1. Aminoglycoside==Gentamicin, Tobramycin, Netilmicin, Amikacin, Streptomycin.

### 2. Cephalosporins=Cefazolin, Cefuroxime, Cefotetan, Ceftriaxone, Ceftazidine.

### 3. Clindamycin

### 4. Macrolides=Erythromycin, Clarithromycin, Azithromycin.

### 5. Metronidazole [0109]

6. Penicillins such as Penicillin, Ampicillin, Nafcillin, Piperacillin. These may be used with or without Aztreonam, Imipenem, or with Beta-lactamase inhibitor including, Ampicillin/sulbactam (Augmentum) or Pipercillin/tazobactam and Beta-lactam=Ceftriaxone, Cefuroxime

### Quinolones=Ciprofloxacin, Ofloxacin, Gatifloxacin or Trovafloxacin

### 8. Tetracyclines=Tetracycline, Doxycycline, or Minocycline

### 9. Trimethoprim-Sulfamethoxazole

### 10. Vancomycin

### 11. Chloramphenicol

### 12. Erythromycin

13. telithromycin a ketolide antibiotic.
14. Ethambutol Myambutol isoniazid pyrazinamide rifabutin
Mycobutin rifampin
Rifadin, Rimactane rifapentine
Priftin Carbapenem antibiotics and amikacin capreomycin
Capastat Sulfate cycloserine
Seromycin ethionamide
Trecator levofloxacin
Levaquin moxifloxacin
Avelox para-aminosalicylic acid
Paser streptomycin.

### Dosing

Selenium should also be administered 200 mg per day if there is inadequate selenium in a patient.

Liposomally encapsulated reduced glutathione (also referred to as liposomal glutathione or liposomal reduced glutathione or liposome-encapsulated glutathione): The preferred dosing schedule of the invention for the treatment of symptoms related to treatment of Klebsiella, CRE diseases or GBS is 800 mg (2 teaspoons) of the invention to be taken twice a day on an empty stomach (that is do not ingest until 30 minutes after eating solid food) and may administered orally or through a nasogastric tube.

1 teaspoon of the invention of oral liposomally encapsulated reduced glutathione reduced contains approximately 420 mg reduced glutathione (“GSH”), and may contain 423 mg reduced glutathione, and 428 mg reduced glutathione.

A preferred mode sets a suggested dose based on body weight. Recommended amounts are for use in the treatment of Klebsiella, CRE diseases or GBS. For best results it is suggested that the invention be used if there is a finding of Klebsiella, CRE diseases or GBS. These doses may also be used if there is a finding of an elevation of a biomarker of Klebsiella, CRE diseases or GBS or as a prophylactic treatment. Gently stir liposomally encapsulated reduced glutathione into the liquid of your choice.

### Determine Individual Dose by Body Weight: for Children

Under 30 lbs: ¼-½ teaspoon=100-200 mg GSH
30-60 lbs: ½-1 teaspoon=210-420 mg GSH
60-90 lbs: ¾-1.5 teaspoon=316 mg-630 GSH
90-120 lbs: 1-2 teaspoon=422-844 mg GSH
120-150 lbs: 1½-3 teaspoon=630-1260 mg GSH
Over 150 lbs: 1½-3 teaspoons=630-1260 mg GSH

The invention should be used on a continuous basis.

Children—should use a dose of liposomally encapsulated reduced glutathione equivalent to 60 mg/Kg of body weight daily in divided doses.

These doses should be continued for the duration of the duration of the illness and for purposes of maintaining adequate glutathione in tissues before, during and after therapy for Klebsiella, CRE diseases or GBS.

The components of this invention can be administered separately or combined in a single capsule or dose.

Because there are hints that Ebola virus, currently in a large epidemic in West Africa, is evolving, the embodiments are reasonably predicted to be useful as compositions for prophylaxis of Ebola on emergence of early systems in conjunction with hydration and electrolyte maintenance pending diagnosis and thereafter as a palliative to support immune system function.

### DETAILED DESCRIPTION OF THE PREFERRED EMBODIMENTS

The methods of manufacture described in Keller et al U.S. Pat. No. 5,891,465, U.S. Pat. No. 6,610,322, and U.S. Pat. No. 6,726,924 and U.S. provisional application No. 60/597,041 by this inventor are adopted herein and into the modes of this invention and can be applied to the examples without undue experimentation. Liposomal formulations preferred in this invention can be purchased from Biozone, Inc. of Pittsburgh, Calif. Reduced glutathione can be b purchased from Sigma-Aldrich of St. Louis, Mo. or from Kyowa Hakko USA, Inc., 767 3^{rd} Ave. No. 9, of New York City, N.Y. 10017 with a Western regional office at 85 Enterprise, Suite 430, Aliso Viejo, Calif. 92656. Liposomally encapsulated reduced glutathione can be purchased from Your Energy Systems, LLC, 555 Bryant St., Suite 305, Palo Alto, Calif. 94301.

### Example 1

Liposomal glutathione Drink or Spray 2500 mg per ounce or form suitable for encapsulation or gel

| | % w/w |
|---|---|
| Deionized Water | 74.4 |
| Glycerin | 15.00 |
| Lecithin | 1.50 |
| Potassium Sorbate | 0.10 |
| (optional spoilage retardant) | |
| Glutathione (reduced) | 8.25 |

A lipid mixture having components lecithin, and glycerin were commingled in a large volume flask and set aside for compounding. Hydroxylated lecithin is the preferred ingredient.

In a separate beaker, a water mixture having water, glycerin, glutathione were mixed and heated to, but not more than, 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% w/w to 8.5% w/w or higher. The methods of manufacture described in Keller et al U.S. Pat. No. 5,891,465, U.S. Pat. No. 6,958,160 and U.S. Pat. No. 7,150,883 and U.S. provisional application No. 60/597,041 are incorporated in this description. Concentrations of liposomally encapsulated glutathione from 3.3% w/w, 4% w/w, 5% w/w, 6% w/w, 7% w/w, 7.5% w/w, 8% w/w, 8.5% w/w or 9% w/w liposomally encapsulated glutathione may be formed and utilized for dosing by decreasing the amounts of glutathione and preplacing the material with an increase in the sterile water concentration.

### Example 1A

Liposomally encapsulated reduced glutathione Drink or Spray 2500 mg Per Ounce or Form Suitable for Encapsulation or Gel: In %, according to w/w: Deionized Water 75, Glycerin 15.00, Lecithin 1.50, Extract Potassium Sorbate 0.10, Glutathione 8.5 (reduced) A lipid mixture having components lecithin, ethyl alcohol and glycerin were commingled in a large volume flask and set aside for compounding. Hydroxylated lecithin is the preferred ingredient.

In a separate beaker, a water mixture having water, glycerin, glutathione were mixed and heated, but not more than, 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. A spoilage retardant such as potassium sorbate or BHT would be added. The solution would be placed in appropriate dispenser for ingestion as a liquid or administration as a spray. Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% w/w to 8.5% w/w or higher. The methods of manufacture described in Keller et al U.S. Pat. No. 5,891,465, U.S. Pat. No. 6,958,160 and U.S. Pat. No. 7,150,883 and U.S. provisional application No. 60/597,041 are incorporated in this description.

Concentrations of liposomally encapsulated glutathione from 3.3% w/w, 4% w/w, 5% w/w, 6% w/w, 7% w/w, 7.5% w/w, 8% w/w, 8.5% w/w or 9% w/w liposomally encapsulated glutathione may be formed and utilized for dosing by decreasing the amounts of glutathione and preplacing the material with an increase in the sterile water concentration.

### Example 2

Embodiment two of the invention includes the incorporation of the fluid liposome (such as that prepared in Example 1A) into a gelatin based capsule to improve the stability, provide a convenient dosage form, and assist in sustained release characteristics of the liposome. The present embodiment relates to the use of glutathione in the reduced state encapsulated into liposomes or formulated as a preliposome formulation and then put into a capsule. The capsule can be a soft gel capsule capable of tolerating a certain amount of water, a two-piece capsule capable of tolerating a certain amount of water or a two-piece capsule where the liposomes are preformed then dehydrated.

The liposome-capsule unit containing biologically encapsulated material can be taken in addition to orally, used for topical unit-of-use application, or other routes of application such as intra-ocular, intranasal, rectal, or vaginal.

The composition of examples 1 and 2 may be utilized in the encapsulated embodiment of this invention.

Gelatin capsules have a lower tolerance to water on their interior and exterior. The usual water tolerance for a soft gel capsule is 10% w/w on the interior. The concentration of water in a liposome formulation can range from 60-90% water. An essential component of the present invention is the formulation of a liposome with a relatively small amount of water, in the range of 5-10% w/w. By making the liposome in a low aqueous system, the liposome is able to encapsulate the biologically active material and the exposure of water to the inside lining of the capsule is limited. The concentration of water should not exceed that of the tolerance of the capsule for which it is intended. The preferred capsule for this invention is one that can tolerate water in the 15-20% w/w range.

The methods described by Keller et al, U.S. Pat. No. 6,726,924 are incorporated in this description.

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. Filling of gel caps on a large scale is best with the rotary die method or others such as the Norton capsule machine.

### Example 3

Liposomally encapsulated S-Nitroso-L-glutathione (GSNO) Drink or Spray 2500 mg per ounce or form suitable for encapsulation or gel

| | % w/w |
|---|---|
| Deionized Water | 74.4 |
| Glycerin | 15.00 |
| Lecithin | 1.50 |
| Potassium Sorbate | 0.10 |
| (optional spoilage retardant) | |
| GSNO | 8.25 |

Another method of Klebsiella (or CRE disease or GBS) treatment is the encapsulation of GSNO. GSNO (S-nitroso-L-glutathione) in either the lecithin or the self forming liposomes of the current invention for use as an anti-Klebsiella (or CRE disease or GBS) therapy. Liposomal encapsulated GSNO, molecular weight 336.3, is 80 mg/ml of liposomal GSNO and the dosing is ½ teaspoon (2.5 ml), to 4 teaspoons (20 ml.) orally twice a day which range includes ½ teaspoon (2.5 ml), 1 teaspoon (5 ml), and ½ teaspoon (2.5 ml), increments up to 4 teaspoons (20 ml.) orally twice a day.

A lipid mixture having components lecithin, and glycerin were commingled in a large volume flask and set aside for compounding.

In a separate beaker, a water mixture having water, glycerin, GSNO were mixed and heated to, but not more than, 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% w/w to 8.5% w/w or higher. The methods of manufacture described in Keller et al U.S. Pat. No. 5,891,465 are incorporated into this description or as described before may be used.

Concentrations of GSNO from 3.3% w/w, 4% w/w, 5% w/w, 6% w/w, 7% w/w, 7.5% w/w, 8% w/w, 8.5% w/w or 9% w/w liposomally encapsulated GSNO may be formed and utilized for dosing by decreasing the amounts of glutathione and preplacing the material with an increase in the sterile water concentration.

### Example 4

Embodiment number four of the present invention includes the creation of liposome suspension using a self-forming, thermodynamically stable liposomes formed upon the adding of a diacylglycerol-PEG lipid to an aqueous solution when the lipid has appropriate packing parameters and the adding occurs above the melting temperature of the lipid. The method described by Keller et al, U.S. Pat. No. 6,610,322 is incorporated into this description.

Most, if not all, known liposome suspensions are not thermodynamically stable. Instead, the liposomes in known suspensions are kinetically trapped into higher energy states by the energy used in their formation. Energy may be provided as heat, sonication, extrusion, or homogenization. Since every high-energy state tries to lower its free energy, known liposome formulations experience problems with aggregation, fusion, sedimentation and leakage of liposome associated material. A thermodynamically stable liposome formulation which could avoid some of these problems is therefore desirable.

The present embodiment prefers liposome suspensions which are thermodynamically stable at the temperature of formation. The formulation of such suspensions is achieved by employing a composition of lipids having several fundamental properties. First, the lipid composition must have packing parameters which allow the formation of liposomes. Second, as part of the head group, the lipid should include polyethyleneglycol (PEG) or any polymer of similar properties which sterically stabilizes the liposomes in suspension. Third, the lipid must have a melting temperature which allows it to be in liquid form when mixed with an aqueous solution.

By employing lipid compositions having the desired fundamental properties, little or no energy need be added when mixing the lipid and an aqueous solution to form liposomes. When mixed with water, the lipid molecules disperse and self-assemble as the system settles into its natural low free energy state. Depending on the lipids used, the lowest free energy state may include small unilamellar vesicle (SUV) liposomes, multilamellar vesicle (MLV) liposomes, or a combination of SUVs and MLVs.

In one aspect, the invention includes a method of preparing liposomes. The method comprises providing an aqueous solution; providing a lipid solution, where the solution has a packing parameter measurement of Pₐ (Pₐ. references the surface packing parameter) between about 0.84 and 0.88, a Pᵥ (Pᵥ references the volume packing parameter) between about 0.88 and 0.93, (See, D. D. Lasic, Liposomes, From Physics to Applications, Elsevier, p. 51 1993), and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolylglycerol-PEG-12, either alone or as one of the lipids in a mixture. The method may further comprise providing an active compound, in this case glutathione (reduced); and combining the active compound with the lipid solution and the aqueous solution.

The low molecular weight in the preferred embodiments more effectively deliver the liposomally encapsulated reduced glutathione in active reduced form as needed and thus result in the surprising effect of the invention. The absorption into cells is a particular advantage of the preferred embodiment of the invention.

### Further Examples 6

### Formulation for Topical Application of Liposomally Encapsulated Reduced Glutathione

A topical cream or lotion containing reduced glutathione in a self-forming liposome sold under the brand name “QuSome” ® by Biozone Laboratories, Inc. of Pittsburgh, Calif. is another preferred embodiment. The Qusome self-forming liposome can be formed containing reduced liposomally encapsulated glutathione in a concentration of 5% reduced glutathione encapsulated in the liposome. Most liposomes use energy provided as heat, sonication, extrusion, or homogenization for their formation, which gives them a high energy state. Some liposome formulations can experience problems with aggregation, fusion, sedimentation and leakage of liposome associated material which this invention seeks to minimize and does minimize. The Qusome is a more thermodynamically stable liposome formulation. The Qusome self-forming liposome is self-forming at room temperature which that the mixing of the lipid and an aqueous lipid containing solution avoids alteration of the contents by heating. The resulting liposome is in a low free energy state so it remains stable and reproducible. The formulation of this embodiment is reviewed in example 3. The methods of manufacture described in Keller et al U.S. Pat. No. 6,958,160 and U.S. Pat. No. 7,150,883 are incorporated in this description. The most important details of that manufacturing are as follows:

The lipids used to form the lipid vesicles and liposomes in the present formulations can be naturally occurring lipids, synthetically made lipids or lipids that are semisynthetic. Any of the art known lipid or lipid like substances can be used to generate the compositions of the present invention. These include, but are not limited to, lecithin, ceramides, phosphatidylethanolamine, phosphotidylcholine, phosphatidylserine, cardiolipin and the like. Such lipid components for the preparation of lipid vesicles are well known in the art, for example see U.S. Pat. No. 4,485,954, and “Liposome Technology”, 2nd Ed, Vol. I (1993) G. Gregoriadis ed., CRC Press, Boca Raton, Fla.

Lipids with these properties that are particularly preferred in the present formulations include phospholipids, particularly highly purified, unhydrogenated lecithin containing high concentrations of phosphotidylcholine, such as that available under the trade name Phospholipon 90 from American Lecithin, or Nattermann Phospholipid, 33 Turner Road, Danbury, Conn. 06813-1908.

In formulating the liposomes, in one aspect, the invention includes a method of preparing liposomes. The method comprises providing an aqueous solution; providing a lipid solution, where the solution has a Pₐ between about 0.84 and 0.88, a Pᵥ between about 0.88 and 0.93, and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolyglycerol-PEG-12, either alone or as one of the lipids in a mixture. The method may further comprise providing an active compound; and combining the active compound with the lipid solution and the aqueous solution.

In another aspect, the invention includes a liposome suspension. The suspension comprises one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The suspension may comprise a single lipid. The lipid may comprise dioleolylglycerol-PEG-12. The suspension may further comprise an active compound, which may be selected from the group described above.

In another aspect, the invention includes a composition for combining with an aqueous solution to form a liposome suspension. The composition comprises one or more lipids, where the lipids as an aggregate have a Pₐ between about 0.84 and 0.88, a Pᵥ, between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The composition may comprise a single lipid. The composition may comprise dioleolylglycerol-PEG 12. The composition may further comprise an active compound selected from the group above. The composition may be provided in a sealed container, where the container also contains an inert gas to prevent oxidative degradation.

In another aspect, the invention includes a method of intravenously administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension intravenously. The method may further comprise providing kinetic energy to the liposome suspension. The method may also include providing the composition in a sealed container containing an inert gas. The PEG chain preferably has a molecular weight between about 300 Daltons and 5000 Daltons. The composition may comprise a single lipid. The lipid may comprise dioleolylglycerol-PEG-12. The active compound may be selected from the group above.

In another aspect, the invention includes a method of solubilizing an active compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing the active compound; providing an aqueous solution; and combining the active compound, the lipid and the aqueous solution to form a liposome suspension. The method may further comprise providing kinetic energy to the liposome suspension. The method may include providing the composition in a sealed container containing an inert gas. The PEG chain preferably has a molecular weight between about 300 Daltons and 5000 Daltons. The composition may comprise, a single lipid. The lipid may comprise dioleolylglycerol-PEG-12. The active compound may be selected from the group above.

In another aspect, the invention includes a method of orally administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension orally in the form selected from the group comprising a two piece hard gelatin capsule, a soft gelatin capsule, or drops.

The compositions may be administered topically, inter-orally, vaginally or rectally.

PEG-12 Glyceryl Dioleate was obtained from Global 7 (New Jersey) for the following formulations. This can be substituted for the lecithin w/w % as needed to accomplish the formulation, or applied as set forth below.

In the following formulations, the “set percentage” w/w % of reduced glutathione is selected from 3.3%, 4%, 5%, 6%, 7%, 7.5%, 8%, 8.5% or 9% or amounts approximately to those percentages.

### Example 5A

### Spontaneous Liposomes for Intravenously Administering Therapeutic Compounds or for a Spray or Drink

A set percentage of reduced glutathione is dissolved in a sufficient amount of the solvent PEG-12 Glyceryl Dioleate, also called dioleolylglycerol-PEG 12, (either referred to as “PEGDO”) and gently mixed for about 5 minutes. A sufficient amount of PEGDO should be about 10% w/w. Deionized water is slowly added to the solution. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste or other flavor-masking ingredients could also be added before the deionized water is brought up to 100% w/w. Although taste ingredients can be added before or after the liposomal encapsulation formulation, the preferable mode is to add flavor or other taste masking ingredients after liposomal encapsulation formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10% w/w. The inventor has preferably used citrus seed extract.

### Example 5B

### Spontaneous Liposomes for Intravenously Administered Therapeutic Compound and as a Drug Solubilization Vehicle for Use in Spray or Drink

A set percentage of reduced glutathione is mixed with a sufficient amount of PEG-12 Glyceryl Dioleate, also called dioleolylglycerol-PEG 12, (either referred to as “PEGDO”) to bring the reduced glutathione into solution by vortexing and sonication for 10 minutes. A sufficient amount of PEGDO should be about 5% w/w. Deionized water is added and gently mixed. Ingredients other than deionized water, the reduced glutathione and the PEGDO may be added such as preferably 0.1% w/w potassium sorbate and then the final amount of deionized water added is that amount which is necessary to have the percentages add up to 100% w/w. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste ingredients or other flavor masking ingredients could also be added before the deionized water is brought up to 100% w/w. Although taste ingredients can be added before or after the liposomal formulation, the preferable mode is to add flavor or other taste masking ingredients after liposomal formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10% w/w. The inventor has preferably used citrus seed extract.

The QuSome self-forming liposome uses polyethyleneglycol (PEG) is a steric stabilizer and the resulting liposome is of a moderate size, 150 nm-250 nm. The combination of 150 nm-250 nm size and the PEG component is known to create long circulating liposomes. The size of the QuSome self-forming liposome allows them to be sterile filtered.

The concentration of liposomally encapsulated glutathione in the liposomes resulting from the Qusome formulation is 5% w/w for topical application. It is possible to use the Qusome technology in creating an oral formulation also and the 8.25% glutathione in w/w concentration encapsulated in the liposome may be used in the oral formulation.

### Examples

### Example 6

The invention is a method of treatment of GBS and CRE diseases, including Klebsiella pneumonia, including direct action against the organism, and a composition for the treatment of Klebsiella pneumoniae (referenced as “Klebsiella” for short) (or CRE disease or GBS) by the direct action of liposomal reduced glutathione.

Liposomal reduced glutathione, particularly that formulated by and sold by Your Energy Systems, LLC of Palo Alto, Calif., would be administered to mammalian patients, upon admission to the ICU, particularly those exhibiting respiratory distress or symptoms or upon receipt of a culture identifying the presence of Klebsiella (or CRE disease or GBS), particularly humans or as a prophylactic to protect neonates. The preferred dosage for a 70 kg patient would be 4 teaspoons daily in an oral formulation having approximately an 8.25% w/w concentration of reduced glutathione in the liposomal formulation. It could be any concentration above 3.3% w/w within the liposomes normally in increments of 0.5% w/w between 3.3% w/w and 9% w/w or higher. There are approximately 423 mg. of reduced glutathione per teaspoon but maybe 420 or 428 mg per teaspoon. Administration may be oral, by inhalation, mucosal, rectal, or intravenous administration.

The dosing applicable for GBS, CRE diseases or Klebsiella is appropriate for Staphylococcus genus, and also for the Escherichia genus including coli, and to diminish the effects of Shigella and shigellosis, Chlamydia, and leishmaniasis.

### Experimental Evidence:

The invention is directed to correcting reduced glutathione deficiencies in lung tissue(s). By increasing the concentration of reduced glutathione intracellularly in affected lung tissues, direct killing action of the Klebsiella organism occurs as reflected in the experiments recited below.

Klebsiella pneumoniae were grown overnight in Tryptic Soy Broth (99 ml of broth and 1 ml of bacteria). The broth containing the K. pneumoniae was divided into equal volumes, centrifuged, and the pellets were washed twice with phosphate buffered saline (PBS). After the final wash, the two pellets were resuspended, pooled, and brought to a final volume of 10 ml. The final concentration of K. pneumonia was ˜2×1010 colony forming units per ml. After diluting this stock to 2×103 and 2×102 colony forming units per ml, 100 μl of these two dilutions were plated onto 100 mm MacConkey Agar Plates. The plates were then misted with 250 μl of liposomal glutathione (182 μl in 100 ml of PBS; Your Energy Systems, LLC, Palo Alto, Calif.) or PBS. After the plates were incubated upside down overnight at 37° C., the number of colony forming units was determined. For the plates misted with PBS, the colony forming units were 200 and 20 for the 2×103 and 2×102 dilutions, respectively. For the plates misted with liposomal glutathione, the colony forming units were 5 and 1 for the 2×103 and 2×102 dilutions, respectively.

Using a standard protocol [Yeligar et al., J. Immunol. 188(8):3648-57 (2012)], control and ethanol-fed male C57BL/6J mice (aged 8-10 weeks; Jackson Laboratory, Bar Harbor, Me.) were given an intra-tracheal inoculation of K. pneumoniae (2×106 colony forming units; 100 μl). At the four hour time point, mice were randomized to treatment with an oral dose of PBS (20 μl) or liposomal glutathione (20 μl of 84.5 mg/ml). At the 20 hour time point, the colony forming units in the lung were determined. For the control mice, the colony forming units of K. pneumoniae in the lung fluid were 40±3 and 21±4 in the PBS and liposomal glutathione treated groups, respectively. For the ethanol-fed mice, the colony forming units of K. pneumoniae were 62±8 and 16±5 in the PBS and liposomal treated groups, respectively. In some studies, mice were randomized to an intranasal treatment of 25 μl per nasal nare of PBS or liposomal glutathione (182 μl in 100 ml of PBS) at the four hour time point. For the control mice, the colony forming units of K. pneumoniae in the lung fluid were 42±5 and 19±6 in the PBS and liposomal glutathione treated groups, respectively. For the ethanol-fed mice, the colony forming units of K. pneumoniae were 74±9 and 14±6 in the PBS and liposomal treated groups, respectively.

### Example 7

### Markers for CRE Diseases Including Klebsiella

Among other markers, a marker for showing Klebsiella in lung tissue and to be combined with the novel method of treatment is to examine reduced glutathione levels in the plasma, bronchoalveolar lavage (BAL) fluid or lung tissue. The inventors propose to initially measure the glutathione levels which normally contains 350-500 micromole (μM) GSH in the lavage fluid or lung tissue, correlate that to an existing CRE or Klebsiella culture, and then examine the glutathione level progression in the lavage or lung tissue in subsequent BAL glutathione determinations to determine the efficacy and dosage of the liposomal glutathione proposed to be used to treat the CRE or Klebsiella.

Another marker for administration of the present invention is to administer liposomally formulated reduced glutathione to individuals with elevations of the cytokine known as transforming growth factor β or transforming growth factor β1 in a blood sample. Transforming growth factor β (TGF-β), which is used to denote both Transforming growth factor β and Transforming growth factor β1, is the most potent and ubiquitous profibrogenic cytokine and its expression is increased in almost all fibrotic diseases. Examples of fibrotic diseases include the fibrosis found in chronic lung diseases described as pulmonary fibrosis, which are associated with chronic lung infection and inflammation. Studies have shown that TGF-β1 decreases intracellular GSH concentration in various types of cells in vitro. Elevated TGF-β can decrease the level of GSH by inhibiting the production and enhance degradation of GCLC, the catalytic subunit of the enzyme responsible for the rate-limiting step in de novo synthesis of reduced glutathione (rGSH). This means that in situations with elevations of TGF-β1 that the building blocks of GSH (such as cysteine found in N-acetyl cysteine, abbreviated NAC) are not as effective as supplying the whole molecule of GSH as is supplied by liposomal reduced glutathione. It has been shown that that liposomal reduced glutathione is 1000 times more effective than NAC in supporting macrophage cell function in the presence of elevated TGF-β1 (Morris 2013). The expected value of measure for TGF-β would be: Normal human subjects were 4.1+/−2.0 ng/ml TGF-beta1 (range, 2.0-12.0 in plasma (1) (2) with >137+/−81 ng/mL in serum considered abnormal. A patient responding appropriately to the treatment with liposomally formulated reduced glutathione would begin to diminish from elevated levels back to normal levels.

### Example 8

### Treatment of Infants with Liposomal Glutathione

The recommended dose of liposomal glutathione for infants is 3 mg/l pound (2.2 Kg) to be taken orally or by nasogastric tube in the infant formula or water. A similar dose may be administered intravenously for cases where oral feeding or nasogastric tube feeding is not possible. A patient example is that a neonate suffering from wheezing was given approximately 6 drops per feeding with 4 feedings per day of concentration of approximately 123 mM liposomal glutathione. 20 Drops is considered one cc. Despite the child being in a high risk category for developing Group B Streptococcus, the child did not develop a GBS infection.

### Example 9

### Markers for Evolving Bacteria Diseases Such as Strep B or Klebsiella

One of the problems of evolving bacterial diseases such as those listed herein is that the rate of culture to determine the disease type may be slower than the propagation within the body. This may unnecessarily and fatally delay treatment or make any cure or diminution of the disease much less effective.

Strep B can be cultured by traditional means, but other potential markers deserve attention.

Among other markers, a marker for showing Klebsiella in lung tissue and to be combined with the novel method of treatment is to examine reduced glutathione levels in the plasma, bronchoalveolar lavage (BAL) fluid or lung tissue. The inventors propose to initially measure the glutathione levels which normally contains 350-500 micromole (μM) GSH in the lavage fluid or lung tissue, correlate that to existing Klebsiella culture, and then examine the glutathione level progression in the lavage or lung tissue in subsequent BAL glutathione determinations to determine the efficacy and dosage of the liposomal glutathione proposed to be used to treat the Klebsiella. A similar approach can be taken with Strep B/GBS infection in seriously affected tissues.

Another marker for administration of the present invention is to administer liposomally formulated reduced glutathione to individuals with elevations of the cytokine known as transforming growth factor β or transforming growth factor β1 in a blood sample. Transforming growth factor β (TGF-β), which is used to denote both Transforming growth factor β and Transforming growth factor β1, is the most potent and ubiquitous profibrogenic cytokine and its expression is increased in almost all fibrotic diseases. Examples of fibrotic diseases include the fibrosis found in chronic lung diseases described as pulmonary fibrosis, which are associated with chronic lung infection and inflammation. Studies have shown that TGF-β1 decreases intracellular GSH concentration in various types of cells in vitro. Elevated TGF-β can decrease the level of GSH by inhibiting the production and enhance degradation of GCLC, the catalytic subunit of the enzyme responsible for the rate-limiting step in de novo synthesis of reduced glutathione (rGSH). This means that in situations with elevations of TGF-β1 that the building blocks of GSH (such as cysteine found in N-acetyl cysteine, abbreviated NAC) are not as effective as supplying the whole molecule of GSH as is supplied by liposomal reduced glutathione. It has been shown that that liposomal reduced glutathione is 1000 times more effective than NAC in supporting macrophage cell function in the presence of elevated TGF-β1 (Morris 2013). The expected value of measure for TGF-β would be: Normal human subjects were 4.1+/−2.0 ng/ml TGF-beta1 (range, 2.0-12.0 in plasma (1) (2) with >137+/−81 ng/mL in serum considered abnormal. A patient responding appropriately to the treatment with liposomally formulated reduced glutathione would begin to diminish from elevated levels back to normal levels.

PCR (polymerase chain reaction) studies to identify DNA sequences for determination of the presence of bacteria in blood or tissue samples such as lung secretion (sputum or lavage of the bronchioles and alveolae during bronchoscopy) can also be used as markers for identifying bacterial infection.

### Example 10

For these diseases, though less preferred, one can provide treatment with plain glutathione 1200 mg 1-4 times per day IV for typical patient presentments. However, 500 mg, 600 mg, 700 mg, 800 mg, 900, mg, 1000 mg, 1100 mg and 1300 mg could also be used. For severe cases, up to 5000 mg IV is recommended 1-4 times per day, meaning 1500 mg, 2000 mg, 2500 mg, 3000 mg, 3500 mg, 4000 mg, 4500 mg, or 5000 mg. Similar doses may be given orally. Intranasal administration of 300-1200 mg, preferably in a dilute saline carrier could also be considered. Thus 300 mg, 400 mg, 500 mg, 600 mg, 700 mg, 800 mg, 900 mg, 1000 mg, 1100 mg, or 1200 mg could be used. An appropriate pharmaceutical buffer such as bicarbonate can be used to adjust pH to a less acidic level.

Thus an aspect of the invention is a method of treatment of patients admitted to an intensive care unit (ICU) of Carbapenem-resistant enterobacteriaceae (CRE) with liposomally formulated reduced glutathione and Vitamin D in a mammalian patient according to the dosages given above.

Another aspect of the invention is a composition of liposomally formulated glutathione and Vitamin D as set forth herein for treatment of patients admitted to an ICU in order to prevent and treat Carbapenem-resistant enterobacteriaceae (CRE) in a mammalian patient according to the dosages given above. The invention can also be prophylactic against CRE diseases and Strep B simultaneously.

Excluded from the claims herein is the joint invention claimed in PCT/US14/16495.

Another aspect of the invention is a composition of liposomally encapsulated S-Nitroso-L-glutathione (GSNO) Drink or Spray for treatment of patients admitted to the ICU with Vitamin D of Carbapenem-resistant enterobacteriaceae (CRE) referenced in this invention.

Another aspect of the invention is the composition of liposomal formulated reduced glutathione for treatment of patients admitted to the ICU for CRE diseases and who require treatment of CRE diseases including Klebsiella, staphylococcus, E. coli, Chlamydia, and leishmaniasis referenced in this invention, or of Streptococcus B.

Another aspect of the invention is the composition of Vitamin D with liposomal formulated reduced glutathione for treatment of patients admitted to the ICU for CRE diseases and who require treatment of CRE diseases including Klebsiella, staphylococcus, E. coli, Chlamydia, and leishmaniasis referenced in this invention, or of Streptococcus B.

Another aspect is evaluating according to the invention biomarkers in association with the compositions, compositions for treatment and methods of treatment in order to assess the effectiveness in a patient of the proposed treatment. Key biomarkers to measure to show effectiveness of the compositions for treatment and methods of treatment are: reduction of elevated TGF-β, examining glutathione levels which are likely deficient and examining the progress of these levels to normal levels in plasma, bronchoalveolar lavage fluid or lung tissue.

### REFERENCES

1. Fitzpatrick A M, Teague W G, Burwell L, Brown M S, Brown L A. Glutathione oxidation is associated with airway macrophage functional impairment in children with severe asthma. Pediatr Res. 2011; 69(2):154-9. http://www.ncbi.nlm.nih.gov/pubmed/20975618
2. Zeevalk G D, Bernard L P, Guilford F T. Liposomal-glutathione provides maintenance of intracellular glutathione and neuroprotection in mesencephalic neuronal cells. Neurochem Res. 2010; 35(10):1575-87. http://www.ncbi.nlm.nih.gov/entrez/query.fcgi?cmd=Retrieve&db=PubMed&dopt=Citation&list_uids=20535554

I claim:

1. A method of prophylaxis and treatment of patients potentially having Streptococcus B or multiple carbapenem-resistant enterobacteriaceae admitted to an intensive care unit comprising:
administering to said patient a dose of Vitamin D and a dose of reduced glutathione stabilized and encapsulated in a liposomal pharmaceutical carrier where the concentration of reduced glutathione in the entrapped aqueous space of the liposomes is at least 123 mM.

2. A composition that can be orally administered to a patient, said composition being reduced glutathione stabilized and encapsulated in a liposomal pharmaceutical carrier capable of being ingested orally, and capable of delivering glutathione (reduced) in a physiologically active state by transfer of the glutathione into patient cells, where the concentration of reduced glutathione in the entrapped aqueous space of the liposomes is at least 123 mM, said composition being for the prophylaxis and treatment of a patient admitted to an intensive care unit to prevent and treat disease symptoms arising from multiple carbapenem-resistant enterobacteriaceae including bacteria selected from the family groups of Klebsiella, Staphylococcus, Clostridium, Shigella, Leishmaniasis, Pneumonia, Escheria, Chlymadia, and Anthrax, or for prophylaxis and treatment of a patient admitted to an intensive care unit to prevent and treat disease symptoms of Streptococcus B

3. A method of measuring the effectiveness of treatment of a patient having disease symptoms arising from carbapenem-resistant enterobacteriaceae, comprising the following steps:
orally administering, to a patient having disease symptoms arising from carbapenem resistant strains of enterobacteriaceae including bacteria selected from the family groups of Klebsiella, Staphylococcus, Clostridium, Shigella, Leishmaniasis, Pneumonia, Escheria, Chlymadia, and Anthrax, a dose of Vitamin D and a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM;
measuring the reduction of elevated TGF-β in blood plasma;
measuring whether the level of said bacteria has fallen; and thereafter continuing to orally administer said glutathione and Vitamin D and re-measuring said level of said bacteria until normal bodily health is achieved.

4. A method of treatment of patients admitted to a hospital with a severe unidentified infection, comprising the following steps:
administering reduced glutathione stabilized and encapsulated in a liposomal pharmaceutical carrier capable of being ingested orally, and capable of delivering glutathione (reduced) in a physiologically active state to improve disease symptoms of said severe unidentified infection by transfer of the glutathione into animal cells, where the concentration of reduced glutathione in the entrapped aqueous space of the liposomes is at least 123 mM; and
administering Vitamin D.

5. A composition of a) reduced glutathione stabilized and encapsulated in a liposomal pharmaceutical carrier capable of being ingested orally, and capable of delivering glutathione (reduced) in a physiologically active state to improve disease symptoms of said severe unidentified infection by transfer of the glutathione into animal cells, where the concentration of reduced glutathione in the entrapped aqueous space of the liposomes is at least 123 mM, and b) Vitamin D for treatment of patients admitted to a hospital with a severe unidentified infection.
